(19) United States
(12) Patent Application Publication
(10) Pub. No.: US 2003/0199533 A1
(43) Pub. Date: Oct. 23, 2003

(54) Novel amino carboxy alkyl derivatives of barbituric acid

(76) Inventor: Kenneth Curry, Vancouver (CA)

Correspondence Address: FISH & NEAVE, 1251 AVENUE OF THE AMERICAS, 50TH FLOOR, NEW YORK, NY, 10020-1105, US

(21) Appl. No.: 10/258,200

(22) PCT Filed: Apr. 18, 2001
(86) PCT No.: PCT/CA01/00503

(30) Foreign Application Priority Data
Apr. 18, 2000 (CA) 2,306,170

(51) Int. Cl. A61K 31/515

(52) U.S. Cl. 514/270

(57) ABSTRACT

The present invention relates to therapeutically active novel amino, carboxy, alkyl derivatives of barbituric acid of Formula (I), wherein at least one of R1, R2, R3 or R4 comprises an NH₂ moiety and at least one of R1, R2, R3 or R4 comprises one COOH moiety. Also provided is a method of preparing compounds of formula (I), and pharmaceutical compositions comprising the compounds. The novel compounds act as modulators of metabotropic glutamate receptors and, as such, are useful in treating diseases of the central nervous system related to the metabotropic glutamate receptor system.

### FIELD OF THE INVENTION

This invention pertains to therapeutically effective amino carboxy derivatives of barbituric acid, a method for preparing the same, pharmaceutical compositions comprising the compounds and a method of treating diseases of the Central Nervous System (CNS) therewith.

### BACKGROUND OF THE INVENTION

The acidic amino acid L-glutamate is recognized as the major excitatory neurotransmitter in the CNS. The receptors that respond to L-glutamate are called excitatory amino acid receptors. The excitatory amino acid receptors are thus of great physiological importance, playing a role in a variety of physiological processes, such as long-term potentiation (learning and memory), the development of synaptic plasticity, motor control, respiratory and cardiovascular regulation, and sensory perception.

Excitatory amino acid receptors are classified into two general types and both are activated by L-glutamate and its analogs. Receptors activated by L-glutamate that are directly coupled to the opening of cation channels in the cell membrane of the neurons are termed “ionotropic.” This type of receptor has been subdivided into at least three subtypes, which are defined by the depolarizing actions of the selective agonists N-Methyl-D-aspartate (NMDA), α-Amino-3-hydroxy-5-methylisoxazole-4-propionic acid (AMPA), and Kainic acid (KA).

The second general type of receptor is the G-protein or second messenger-linked “metabotropic” excitatory amino acid receptor. This second type is coupled to multiple second messenger systems that lead to enhanced phosphoinositide hydrolysis, activation of phospholipase D, increases or decreases in cAMP formation, and changes in ion channel function (Schoepp and Conn, Trends in Pharmacological Science, 14:13, 1993). Both types of receptors appear not only to mediate normal synaptic transmission along excitatory pathways but also to participate in the modification of synaptic connections during development and throughout life.

So far eight different clones of the G-protein-coupled mGluRs have been identified (Knopfel et al., 1995, J Med. Chem., 38, 1417-1426). These receptors function to modulate the presynaptic release of L-glutamate, and the postsynaptic sensitivity of the neuronal cell to L-glutamate excitation. Based on pharmacology, sequence homology and the signal transduction pathway that they activate, the mGluRs have been subclassified into three groups. The mGluR1 and mGluR5 receptors form group I. They are coupled to hydrolysis of phosphatidylinositol (PI) and are selectively activated by (RS)-3,5-dihydroxyphenylglycine (Brabet et al., Neuropharmacology, 34, 895-903, 1995). Group II comprises mGluR2 and mGluR3 receptors. They are negatively coupled to adenylate cyclase and are selectively activated by (2S,1′R,2′R,3′R)-2-(2,3-dicarboxycyclopropyl)glycine (DCG-IV; Hayashi et al., Nature, 366, 687-690, 1993). Finally, the mGluR4, mGluR6, mGluR7 and mGluR8 receptors belong to group III. They are also negatively coupled to adenylate cyclase and are selectively activated by (S)-2-amino-4-phosphonobutyric acid (L-AP4; Knopfel et al., 1995, J. Med. Chem., 38, 1417-1426).

Agonists and antagonists of these receptors are believed useful for the treatment of acute and chronic neurodegenerative conditions, and as antipsychotic, anticonvulsant, analgesic, anxiolytic, antidepressant, and anti-emetic agents. Antagonists and agonists of neural receptors are classified as selective for a particular receptor or receptor subtype, or as non-selective. Antagonists may also be classified as competitive or non-competitive. While competitive and non-competitive antagonists act on the receptors in a different manner to produce similar results, selectivity is based upon the observations that some antagonists exhibit high levels of activity at a single receptor type, and little or no activity at other receptors. In the case of receptor-specific diseases and conditions, the selective agonists and antagonists are of the most value.

Compounds such as L-glutamate, quisqualate and ibotenate are known to act as non-selective agonists on the mGluRs, while selective ionotropic glutamate receptor agonists such as NMDA, AMPA and kainate have little effect on these receptors. Recently a few compounds without activity at the ionotropic glutamate receptors but with activity at the metabotropic receptors have been identified. These include trans-ACPD (trans (1S,3R-1-aminocyclopentane-1,3-dicarboxylic acid), the partial agonist L-AP3 (L-2-amino-3-phosphonopropionic acid; Palmer, E., Monaghan, D. T. and Cotman, C. W. Eur. J. Pharmacol. 166, 585-587, 1989; Desai, M. A. and Conn, P. J. Neuroscience Lett. 109, 157-162, 1990; Schoepp, D. D. et al., J. Neurochemistry 56, 1789-1796, 1991; Schoepp D. D. and Johnson B. G. J. Neurochemistry 53, 1865-1613, 1989), L-AP4 (L-2-amino-4-phosphonobutyrate) which is an agonist at the mGluR4 receptor (Thomsen C. et al., Eur. J. Pharmacol. 227, 361-362, 1992) and some of the isomers of CCG (2-(carboxycyclopropyl)glycines) especially L-CCG-I and L-CCG-II (Hayashi, Y. et al., Br. J Pharmacol. 107, 539-543, 1992).

Very few selective antagonists at the mGluRs have been reported. However some phenylglycine derivatives, S-4CPG (S-4-carboxyphenylglycine), S-4C3HPG (S-4-carboxy-3-hydroxyphenylglycine) and S-MCPG (S-α-methyl-4-carboxyphenylglycine) have been reported to antagonize trans-ACPD-stimulated phosphoinositide hydrolysis and thus possibly act as antagonists at mGluR1 and mGluR5 subtypes (Thomsen, C. and Suzdak, P, Eur. J. Pharmacol. 245, 299, 1993).

Research directed towards mGluRs is beginning to show that mGluRs may be implicated in a number of normal as well as pathological mechanisms in the brain and spinal cord. For example, activation of these receptors on neurons can: influence levels of alertness, attention and cognition; protect nerve cells from excitotoxic damage resulting from ischemia, hypoglycemia and anoxia; modulate the level of neuronal excitation; influence central mechanisms involved in controlling movement; reduce sensitivity to pain; reduce levels of anxiety.

The use of compounds active at the mGluRs for the treatment of epilepsy is corroborated by investigations of the influence of trans-ACPD on the formation of convulsions (Sacaan and Schoepp, Neuroscience Lett. 139, 77, 1992) and that phosphoinositide hydrolysis mediated via mGluR is increased after kindling experiments in rats (Akiyama et al. Brain Res. 569, 71, 1992). Trans-ACPD has been shown to increase release of dopamine in the rat brain, which indicates that compounds acting on the mGluRs might be usable for the treatment of Parkinson's disease and Huntington's Chorea (Sacaan et al., J. Neurochemistry 59, 245, 1992).

Trans-ACPD has also been shown to be a neuroprotective agent in a medial cerebral artery occlusion (MCAO) model in mice (Chiamulera et al. Eur. J Pharmacol. 215, 353, 1992), and it has been shown to inhibit NMDA-induced neurotoxicity in nerve cell cultures (Koh et al., Proc. Natl. Acad. Sci. USA 88, 9431, 1991). The mGluR-active compounds are also implicated in the treatment of pain. This is proved by the fact that antagonists at the metabotropic glutamate receptors antagonizes sensory synaptic response to noxious stimuli of thalamic neurons (Eaton, S. A. et al., Eur. J. Neuroscience, 5, 186, 1993).

The use of compounds active at the mGluRs for treatment of neurological diseases such as senile dementia have also been indicated by the findings of Zheng and Gallagher (Neuron 9, 163, 1992) and Bashir et al. (Nature 363, 347, 1993) who demonstrated that activation of mGluRs is necessary for the induction of long term potentiation (LTP) in nerve cells (septal nucleus, hippocampus) and the finding that long term depression is induced after activation of metabotropic glutamate receptors in cerebellar granule cells (Linden et al. Neuron 7, 81, 1991).

Thus, compounds that demonstrate either activating or inhibiting activity at mGluRs have therapeutic potential for the treatment of neurological disorders. These compounds have application as new drugs to treat both acute and chronic neurological disorders, such as stroke and head injuries; epilepsy; movement disorders associated with Parkinson's disease and Huntington's chorea; pain; anxiety; AIDS dementia; and Alzheimer's disease. Since the GluRs can influence levels of alertness, attention and cognition; protect nerve cells from excitotoxic damage resulting from ischemia, hypoglycemia and anoxia; modulate the level of neuronal excitation; influence central mechanisms involved in controlling movement; reduce sensitivity to pain; and reduce levels of anxiety, these compounds can also be used to influence these situations and also find use in learning and memory deficiencies such as senile dementia. mGluRs may also be involved in addictive behavior, alcoholism, drug addiction, sensitization and drug withdrawal (Science, 280:2045, 1998), so compounds acting at mGluRs might also be used to treat these disorders.

The current pharmaceutical options for treating neurological disorders tend to be very general and non-specific in their actions in that, although they may reduce the clinical symptoms associated with a specific neurological disorder, they may also negatively impact normal function of the central nervous system of patients. Thus new cellular targets and drugs that are more specific in their actions require identification and development and a need remains for chemical compounds that demonstrate specific binding characteristics towards mGluRs.

### SUMMARY OF THE INVENTION

It is an object of the present invention to provide novel compounds which are amino carboxy derivatives of barbituric acid that demonstrate activity at the various metabotropic glutamate receptors. In one aspect of the present invention there is provided a compound of Formula I and stereoisomers thereof:

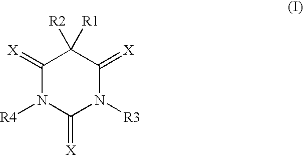

wherein:

R1 and R2 are same or different and selected from the group comprising H, NH₂, COOH and (CH₂)nCR5WY, wherein: n=0-5, W is H or —COOH, Y is H or NH₂, R5 is H, alkyl, aryl, or (CH₂)mR6, wherein: m=0-5 and R6 is H, carboxyl, phosphono, phosphino, sulfono, sulfino, borono, tetrazol, or isoxazol;

R3 and R4 are same or different and selected from the group comprising H, alkyl, aryl, acyl, CH₂COOH, NH₂ and —(CH₂)n′CHCO₂H NH₂, wherein: n′ is 0-5;

and X is O or S;

with the proviso, that at least one of R1, R2, R3 or R4 comprises an NH₂ moiety and at least one of R1, R2, R3 or R4 comprises one COOH moiety.

### DETAILED DESCRIPTION OF THE INVENTION

The terms and abbreviations used in the instant examples have their normal meanings unless otherwise designated. For example “° C.” refers to degrees Celsius; “N” refers to normal or normality; “mmol” refers to millimole or millimoles; “g” refers to gram or grams; “mL” means milliliter or milliliters; “M” refers to molar or molarity; “MS” refers to mass spectrometry; “IR” refers to infrared spectroscopy; and “NMR” refers to nuclear magnetic resonance spectroscopy.

Alkyl: refers to a saturated straight chain, branched or cyclic hydrocarbon group. Typical alkyl groups include methyl, ethyl, propyl, isopropyl, cyclopropyl, butyl, isobutyl, t-butyl, cyclobutyl, pentyl, isopentyl, cyclopentyl, hexyl, cyclohexyl and the like.

Aryl: refers to an unsaturated mono or polycyclic hydrocarbon group having a conjutated π electron system. Typical aryl groups include, but not limited to, penta-2,4-diene, phenyl, naphthyl, anthracyl, azulenyl, indacenyl, and the like.

As would be understood by the skilled artisan throughout the synthesis of the compounds of Formula I, it may be necessary to employ an amino-protecting group or a carboxy-protecting group in order to reversibly preserve a reactively susceptible amino or carboxy functionality while reacting other functional groups on the compound.

Examples of such amino-protecting groups include formyl, trityl, phthalimido, trichloroacetyl, chloroacetyl, bromoacetyl, iodoacetyl, and urethane-type blocking groups such as benzyloxycarbonyl, 4-phenylbenzyloxycarbonyl, 2-methylbenzyloxycarbonyl, 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, t-butoxycarbonyl, 2-(4-xenyl)-isopropoxycarbonyl, 1,1-diphenyleth-1-yloxycarbonyl, 1,1-diphenylprop-1-yloxycarbonyl, 2-phenylprop-2-yloxycarbonyl, 2-(p-toluyl)-prop-2-yloxycarbonyl, cyclopentanyloxy-carbonyl, 1-methylcyclopentanyloxycarbonyl, cyclohexanyloxycarbonyl, 1-methylcyclohexanyloxycarbonyl, 2-methylcyclohexanyloxycarbonyl, 2-(4-toluylsulfono)-ethoxycarbonyl, 2-(methylsulfono)ethoxycarbonyl, 2-(triphenylphosphino)-ethoxycarbonyl, fluorenylmethoxycarbonyl (“FMOC”), 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyloxycarbonyl, 2,2,2-trichloroethoxycarbonyl, 2-ethynyl-2-propoxycarbonyl, cyclopropylmethoxycarbonyl, 4-(decycloxy)benzyloxycarbonyl, isobomyloxycarbonyl, 1-piperidyloxycarbonlyl and the like; benzoylmethylsulfono group, 2-nitrophenylsulfenyl, diphenylphosphine oxide and like amino-protecting groups. The species of amino-protecting group employed is not critical so long as the derivatized amino group is stable to the condition of subsequent reaction(s) on other positions of the intermediate molecule and can be selectively removed at the appropriate point without disrupting the remainder of the molecule including any other amino-protecting group(s). Preferred amino-protecting groups are t-butoxycarbonyl (t-Boc), allyloxycarbonyl and benzyloxycarbonyl (CbZ). Further examples of these groups are found in E. Haslam in Protective Groups in Organic Synthesis; McOmie, J. G. W., Ed. 1973, at Chapter 2; and Greene, T. W. and Wuts, P. G. M., Protective Groups in Organic Synthesis, Second edition; Wiley-Interscience: 1991; Chapter 7.

Examples of carboxyl-protecting groups include methyl, p-nitrobenzyl, p-methylbenzyl, p-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,2′,4,4′-tetramethoxybenzhydryl, t-butyl, t-amyl, trityl, 4-methoxytrityl, 4,4′-dimethoxytrityl, 4,4′,4″-trimethoxytrityl, 2-phenylprop-2-yl, trimethylsilyl, t-butyldimethylsilyl, phenacyl, 2,2,2-trichloroethyl, β-(di(n-butyl)methylsilyl)ethyl, p-toluenesulfonoethyl, 4-nitrobenzylsulfonoethyl, allyl, cinnamyl, 1-(trimethylsilylmethyl)prop-1-en-3-yl and like moieties. Preferred carboxyl-protecting groups are allyl, benzyl and t-butyl. Further examples of these groups are found in E. Haslam, supra, at Chapter 5; and T. W. Greene and P. G. M. Wuts, supra, at Chapter 5.

The present invention provides a compound of the formula (I):

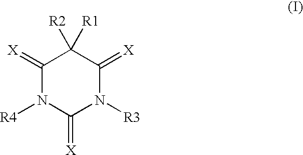

wherein:

R1 and R2 can be the same or different and selected from the group comprising H, NH₂, COOH and (CH₂)nCR5WY, wherein: n=0-5, W is H or COOH, Y is H or NH₂, R5 is H, alkyl, aryl, or (CH₂)mR6, wherein: m=0-5 and R6 is H, carboxyl, phosphono, phosphino, sulfono, sulfino, borono, tetrazol, or isoxazol;

R3 and R4 can be the same or different and selected from the group comprising H, alkyl aryl acyl, CH₂COOH, NH₂ and —(CH₂)n′CHCO₂H NH₂, wherein: n′ is 0-5;

and X is O or S;

with the proviso, that at least one of R1, R2, R3 or R4 comprises an NH₂ moiety and at least one of R1, R2, R3 or R4 comprises one COOH moiety.

The preferred compounds of the present invention include, but are not limited to the following:

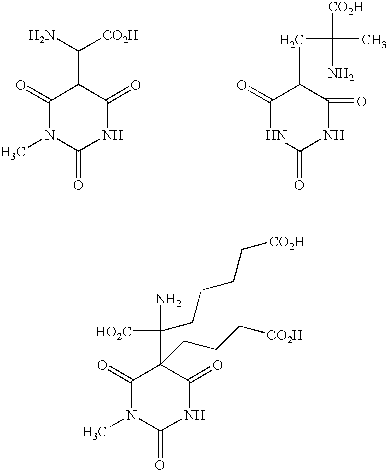

The present invention includes the pharmaceutically acceptable salts of the compounds defined by Formula I. A compound of this invention can possess a sufficiently acidic, a sufficiently basic, or both functional groups, and accordingly react with any of a number of organic and inorganic bases, and inorganic and organic acids, to form a pharmaceutically acceptable salt.

The term “pharmaceutically acceptable salt” as used herein, refers to salts of the compounds of the above formula which are substantially non-toxic to living organisms. Typical pharmaceutically acceptable salts include those salts prepared by reaction of the compounds of the present invention with a pharmaceutically acceptable mineral or organic acid or an organic or inorganic base. Such salts are known as acid addition and base addition salts.

Acids commonly employed to form acid addition salts are inorganic acids such as hydrochloric acid, hydrobromic acid, hydriodic acid, sulfuric acid, phosphoric acid, and the like, and organic acids such as p-toluenesulfonic acid, methanesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate malonate, succinate, suberate, sebacate, fumarate, maleate, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, napththalene-2-sulfonate, mandelate and the like. Preferred pharmaceutically acceptable acid addition salts are those formed with mineral acids such as hydrochloric acid and hydrobromic acid, and those formed with organic acids such as maleic acid and methanesulfonic acid.

Salts of amine groups may also comprise quarternary ammonium salts in which the amino nitrogen carries a suitable organic group such as an alkyl, alkenyl, alkynyl, or aralkyl moiety.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred.

It should be recognized that the particular counterion forming a part of any salt of this invention is usually not of a critical nature, so long as the salt as a whole is pharmacologically acceptable and as long as the counterion does not contribute undesired qualities to the salt as a whole. This invention further encompasses the pharmaceutically acceptable solvates of the compounds of Formula I. Many of the Formula I compounds can combine with solvents such as water, methanol, ethanol and acetonitrile to form pharmaceutically acceptable solvates such as the corresponding hydrate, methanolate, ethanolate and acetonitrilate.

The compounds of the present invention have multiple asymmetric (chiral) centers. As a consequence of these chiral centers, the compounds of the present invention occur as racemates, mixtures of enantiomers and as individual enantiomers, as well as diastereomers and mixtures of diastereomers. All asymmetric forms, individual isomers and combinations thereof, are within the scope of the present invention.

The prefixes “R” and “S” are used herein as commonly used in organic chemistry to denote the absolute configuration of a chiral center, according to the Cahn-Ingold-Prelog system. The stereochemical descriptor R (rectus) refers to that configuration of a chiral center with a clockwise relationship of groups tracing the path from highest to second-lowest priorities when viewed from the side opposite to that of the lowest priority group. The stereochemical desciptor S (sinister) refers to that configuration of a chiral center with a counterclockwise relationship of groups tracing the path from highest to second-lowest priority when viewed from the side opposite to the lowest priority group. The priority of groups is decided using sequence rules as described by Cahn et al., Angew. Chem., 78, 413-447, 1966 and Prelog, V. and Helmchen, G.; Angew. Chem. Int. Ed. Eng., 21, 567-583, 1982).

In addition to the R,S system used to designate the absolute configuration of a chiral center, the older D-L system is also used in this document to denote relative configuration, especially with reference to amino acids and amino acid derivatives. In this system a Fischer projection of the compound is oriented so that carbon-1 of the parent chain is at the top. The prefix “D” is used to represent the relative configuration of the isomer in which the functional (determining) group is on the right side of the carbon atom at the chiral center and “L”, that of the isomer in which it is on the left.

As would be expected, the stereochemistry of the Formula I compounds is critical to their potency as agonists or antagonists. The relative stereochemistry is established early during synthesis, which avoids subsequent stereoisomer separation problems later in the process. Further manipulation of the molecules then employs stereospecific procedures so as to maintain the preferred chirality. The preferred methods of this invention are the methods employing those preferred compounds.

Non-toxic metabolically-labile ester and amide of compounds of Formula I are ester or amide derivatives of compound of Formula I that are hydrolyzed in vivo to afford said compounds of Formula I and a pharmaceutically acceptable alcohol or amine. Examples of metabolically-labile esters include esters formed with (1-6C) alkanols in which the alkanol moiety may be optionally substituted by a (1-8C) alkoxy group, for example methanol, ethanol, propanol and methoxyethanol. Examples of metabolically-labile amides include amides formed with amines such as methylamine.

According to another aspect, the present invention provides a process for the preparation of a compound of Formula I, in which R1 and R2 are not both H or a pharmaceutically acceptable metabolically-labile ester or amide thereof, or a pharmaceutically acceptable salt thereof, which comprises:

(a) hydrolyzing a compound of Formula: II

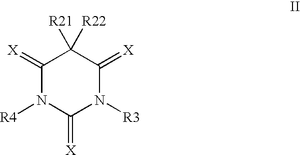

wherein: R21 and R22 can be the same or different and selected from the group:

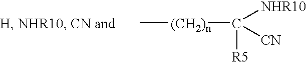

wherein: n=0-5,

R3, R4, R5 are as defined above, R10 represents a hydrogen atom or an acyl group. Preferred values for R10 are hydrogen and (2-6C) alkanoyl groups, such as acetyl; or

(b) by hydrolyzing a compound of Formula: III

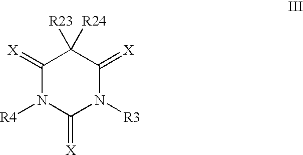

wherein: R23 and R24 can be the same or different and selected from:

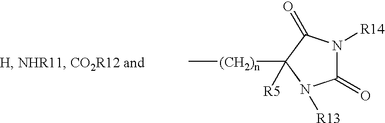

wherein: n=0-5,

R3, R4, R5 are as defined above, R11 represents a hydrogen atom or a nitrogen protecting group and R12 represents a hydrogen atom or a carboxyl protecting group or a salt thereof;

R13 and R14 each independently represent a hydrogen atom, a (2-6 C) alkanoyl group, a (1-4 C) alkyl group, a (3-4 C) alkenyl group or a phenyl (1-4 C) alkyl group in which the phenyl is unsubstituted or substituted by halogen, (1-4 C) alkyl or (1-4 C) alkoxy, or a salt thereof; or

(c) by deprotecting a compound of Formula: IV

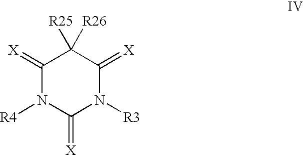

wherein: R25 and R26 can be the same or different and selected from:

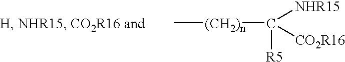

wherein: n=0-5,

R3, R4, R5 are as defined above, R15 represents a hydrogen atom or a nitrogen protecting group and R16 represents a hydrogen atom or a carboxyl protecting group or a salt thereof;

(d) whereafter, if necessary and/or desired:

(i) resolving the compound of Formula I;

(ii) converting the compound of Formula I into a non-toxic metabolically labile ester or amide thereof; and/or;

(iii) converting the compound of Formula I or a non-toxic metabolically labile ester or amide thereof into a pharmaceutically acceptable salt thereof.

The protection of carboxylic acid and amine groups is generally described in McOmie, Protecting Groups in Organic Chemistry, Plenum Press, NY, 1973, and Greene and Wuts, Protecting Groups in Organic Synthesis, 2nd. Ed., John Wiley & Sons, NY, 1991. Examples of carboxyl protecting groups include alkyl groups such as methyl, ethyl, t-butyl and t-amyl; aralkyl groups such as benzyl, 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, benzhydryl and trityl; silyl groups such as trimethylsilyl and t-butyldimethylsilyl; and allyl groups such as allyl and 1-(trimethylsilylmethyl)prop-1-en-3-yl. Examples of amine protecting groups include acyl groups, such as groups of formula R^{11a} CO in which R^{11a} represents (1-6C) alkyl, (3-10C) cycloalkyl, phenyl(1-6C) alkyl, phenyl, (1-6C) alkoxy, phenyl(1-6C) alkoxy, or a (3-10C) cycloalkoxy, wherein a phenyl group may optionally be substituted by one or two substituents independently selected from amino, hydroxy, nitro, halogeno, (1-6C) alkyl, (1-6C) alkoxy, carboxyl, (1-6C) alkoxycarbonyl, carbamoyl, (1-6C) alkanoylamino, (1-6C) alkylsulphonylamino, phenylsulphonylamino, toluenesulphonylamino, and (1-6C) fluoroalkyl.

The compounds of Formula II are conveniently hydrolyzed in the presence of an acid, such as hydrochloric acid or sulfuric acid, or a base, such as an alkali metal hydroxide, for example sodium hydroxide. The hydrolysis is conveniently performed in an aqueous solvent such as water and at a temperature in the range of 50 to 200° C.

The compounds of Formula III are conveniently hydrolyzed in the presence of a base, for example an alkali metal hydroxide such as lithium, sodium or potassium hydroxide, or an alkaline earth metal hydroxide such as barium hydroxide. Suitable reaction media include water. The temperature is conveniently in the range of from 50 to 150° C.

The compounds of Formula IV may be deprotected by a conventional method. Thus, an alkyl carboxyl protecting group may be removed by hydrolysis. The hydrolysis may conveniently be performed by heating the compound of Formula V in the presence of either a base, for example an alkali metal hydroxide such as lithium, sodium or potassium hydroxide, or an alkaline metal hydroxide, such as barium hydroxide, or an acid such as hydrochloric acid. The hydrolysis is conveniently performed at a temperature in the range from 10 to 300° C. An aralkyl carboxyl protecting group may conveniently be removed by hydrogenolysis. The hydrogenolysis may conveniently be effected by reacting the compound of Formula V with hydrogen in the presence of a Group VIII metal catalyst, for example a palladium catalyst such as palladium on charcoal. Suitable solvents for the reaction include alcohols such as ethanol. The reaction is conveniently performed at a temperature in the range from 0 to 100° C. An acyl, amine protecting group is also conveniently removed by hydrolysis, for example as described for the removal of an alkyl carboxyl protecting group.

The compounds of Formula II, wherein R21 is:

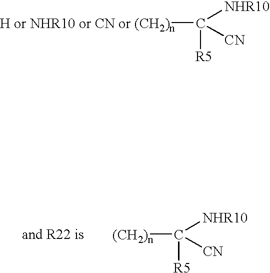

may be prepared by reacting a compound of Formula V with an alkali metal cyanide, such as lithium, sodium or potassium cyanide, and an ammonium halide, such as ammonium chloride, conveniently in the presence of ultrasound. Thus, the ammonium halide is mixed with chromatography grade alumina in the presence of a suitable diluent such as acetonitrile. The mixture is then irradiated with ultrasound, whereafter the compound of Formula V is added, and the mixture is again irradiated. The alkali metal cyanide is then added, followed by further treatemnt with ultrasound.

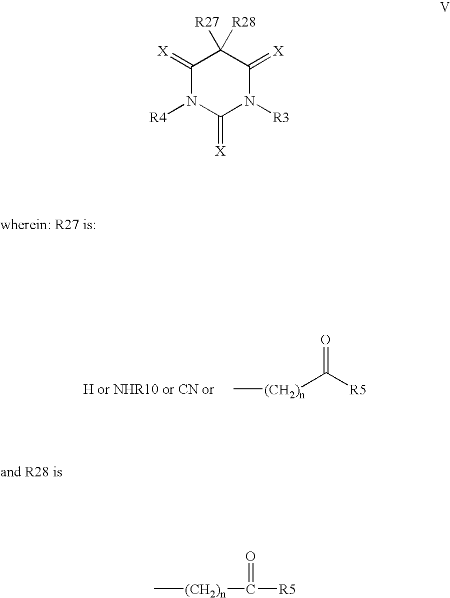

wherein: n=0-5,

Individual isomers of compounds of Formula II may be made by reacting a compound of the Formula V with the stereoisomers of the chiral agent (S) and (R)-phenylglycinol and a reactive cyanide such as trimethylsilyl cyanide.

The compounds of Formula III, wherein R23 is

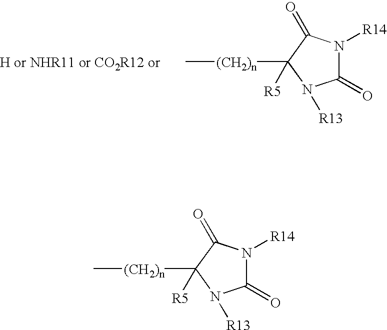

may be prepared by reacting a compound of Formula VI with an alkali metal cyanide, such as lithium, sodium or potassium cyanide, and ammonium carbonate or ammonium carbamate. Convenient solvents include water, dilute ammonium hydroxide, alcohols such as methanol, aqueous methanol and aqueous ethanol. Conveniently the reaction is performed at a temperature in the range of from 10 to 150° C. If desired, the compounds of Formula III may then be N-alkylated, for example using an appropriate compound of formula R3 Cl and/or R4 Cl.

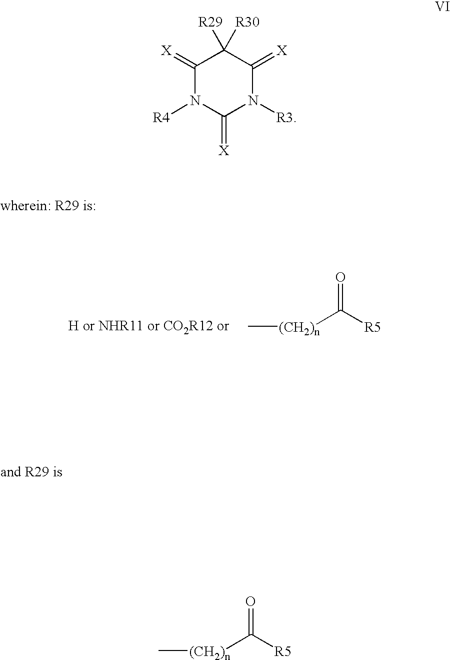

wherein: n=0-5,

Compounds of the Formula V or VI may be prepared from compounds of the Formula VII

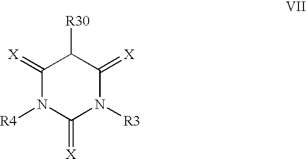

by alkylation or acylation with an appropriate agent selected from the group comprising HC(OC₂H₅)₃, (CH₃CO)₂O and

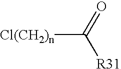

Wherein: R29 is H or NHR10 or CN or NHR11 or CO₂R, n is 0-5 and R31 is H, alkyl (1-3C), aryl, alkoxy (1-4 C),

Compounds of Formula VII may be commercially available or may be prepared by the standard reactions known to a worker skilled in the relevant art.

Compunds of Formula I, wherein both R1 and R2 are H at the same time, can be prepared from barbituric acid and derivatives and precursors thereof by the standard reactions known to a worker skilled in the relevant art.

Many of the intermediates described herein, for example the compounds of Formula II, III and IV are believed to be novel, and are provided as further aspects of the present invention.

The Formula I compounds of the present invention are agonists or antagonists at certain metabotropic excitatory amino acid receptors (mGluRs). Therefore, another aspect of the present invention is a method of affecting mGluRs in mammals, which comprises administering to a mammal requiring modulated excitatory amino acid neurotransmission a pharmacologically-effective amount of a compound of Formula I. The term “pharmacologically-effective amount” is used to represent an amount of the compound of the invention that is capable of affecting the mGluRs. By affecting, a compound of the invention is acting as an agonist or antagonist. When a compound of the invention acts as an agonist, the interaction of the compound with the excitatory amino acid receptor mimics the response of the interaction of this receptor with its natural ligand (i.e. L-glutamate). When a compound of the invention acts as an antagonist, the interaction of the compound with the excitatory amino acid receptor blocks the response of the interaction of this receptor with its natural ligand (i.e. L-glutamate).

The particular dose of compound administered according to this invention will, of course, be determined by the particular circumstances surrounding the case, including the compound administered, the route of administration, the particular condition being treated, and similar considerations. The compounds can be administered by a variety of routes including oral, rectal, transdermal, subcutaneous, intravenous, intramuscular, or intranasal routes. Alternatively, the compound may be administered by continuous infusion. A typical daily dose will contain from about 0.001 mg/kg to about 100 mg/kg of the active compound of this invention. Preferably, daily doses will be about 0.05 mg/kg to about 50 mg/kg, more preferably from about 0.1 mg/kg to about 20 mg/kg.

A variety of physiological functions have been shown to be influenced by excessive or inappropriate stimulation of excitatory amino acid transmission. The Formula I compounds of the present invention are believed (through their interactions at the mGluRs) to have the ability to treat a variety of neurological disorders in mammals associated with excessive or inappropriate stimulation of excitatory amino acid transmission. Such disorders include, but are not limited to, acute neurological disorders such as cerebral deficits subsequent to cardiac bypass surgery and grafting, cerebral ischemia (e.g. stroke and cardiac arrest), spinal cord trauma, head trauma, perinatal hypoxia, and hypoglycemic neuronal damage. The Formula I compounds are believed to have the ability to treat a variety of chronic neurological disorders, such as, but not limited to Alzheimer's disease, Huntington's Chorea, amyotrophic lateral sclerosis, AIDS-induced dementia, ocular damage and retinopathy, cognitive disorders, and idiopathic and drug-induced Parkinson's. The present invention also provides methods for treating these disorders which comprises administering to a patient in need thereof an effective amount of a compound of Formula I.

The Formula I compounds of the present invention (through their interactions at the mgluRs) are also believed to have the ability to treat a variety of other neurological disorders in mammals that are associated with glutamate dysfunction, including, but not limited to, muscular spasms, convulsions, migraine headaches, urinary incontinence, psychosis, drug tolerance, withdrawal, and cessation (i.e. opiates, benzodiazepines, nicotine, cocaine, or ethanol), smoking cessation, anxiety and related disorders (e.g. panic attack), emesis, brain edema, chronic pain, sleep-disorders, Tourette's syndrome, attention deficit disorder, and tardive dyskinesia. Therefore, the present invention also provides methods for treating these disorders which comprise administering to a patient in need thereof an effective amount of the compound of Formula I.

The Formula I compounds of the present invention (through their interactions at the mgluRs) are also believed to have the ability to treat a variety of psychiatric disorders, such as, but not limited to, schizophrenia, anxiety and related disorders (e.g. panic attack), depression, bipolar disorders, psychosis, and obsessive compulsive disorders. The present invention also provides methods for treating these disorders which comprises administering to a patient in need thereof an effective amount of a compound of Formula I.

Functional Assays Employing Cloned Subtypes of Metabotropic Receptors

In order to identify agonist or antagonist activities of a compound a appropriate functional assay using recombinant metabotropic glutamate receptors, adenylate cyclase activity or phosphatidylinositol hydrolysis, can be performed substantially as before using standard procedures.

Pharmacological Demonstration of Certain Compounds at Representative mGlu Receptor Subtypes Expressed in Chinese Hamster Ovary (CHO) Cells.

The Chinese hamster ovary cell lines expressing mGlul1α, mGlu 2 and mGlu 4a receptors have been described previously (Amarori and Nakanishi, Neuron 8, 757-765, 1992; Tanabe et al., Neuron 8, 169-179, 1992, and J. Neurochem. 63, 2038-2047, 1993). They are maintained at 37° C. in a humidified 5% CO₂ incubator in Dubecco's Modified Eagle Medium (DMEM) containing a reduced concentration of (S)-glutamine (2 mM) and are supplemented with 1% proline, penicillin (100 U/mL), streptomycin (100 mg/mL) and 10% dialyzed fetal calf serum (all GIBCO, Paisley). Two days before assay 1.8×10⁶ cells are divided into the wells of 24 well plates.

Phosphatidyl inositol (PI) hydrolysis is measured as described previously (Hayashi et al., Nature 366, 687-690,1992, and J. Neuroscience 14, 3370-3377, 1994). Briefly, the cells are labeled with [³H]inositol (2 μCi/mL) 24 h prior to the assay. For agonist assays, the cells are incubated with ligand dissolved in phosphate-buffered saline (PBS)-LiCl for 20 min, and agonist activity is determined by measurement of the level of ³H-labeled mono-, bis- and tris-inositol phosphates by ion-exchange chromatography. For antagonist assays, the cells are preincubated with the ligand dissolved in PBS-LiCl for 20 min prior to incubation with ligand and 10 μM (S)-Glu for 20 min. The antagonist activity is then determined as the inhibitory effect of the (S)-Glu mediated response. The assay of cyclic AMP formation is performed as described previously (Hayashi et al., 1992, 1994). Briefly, the cells are incubated for 10 min in PBS containing the ligand and 10 μM forskolin and 1 mM 3-isobutyl-1-methylxanthine (IBMX) (both Sigma, St. Louis, Mo., USA). The agonist activity is then determined as the inhibitory effect of the forskolin-induced cyclic AMP formation. For antagonist assay, the cells are preincubated with ligand dissolved in PBS containing 1 mM IBMX for 20 min prior to a 10 min incubation in PBS containing the ligand, 20 μM(mGlu₂) or 50 μM (mGlu₄ₐ) (S)-Glu, 10 μM forskolin and 1 mM IBMX.

All experiments can be performed in triplicate and the results are given as mean±S.E.M. of at least three independent experiments. Antagonist potency can be calculated from the Gaddum equation KB=[B]/(DR−1) (Lazareno and Birdsall, Trends Phamacol. Sci. 14, 237-239,1993), where the dose-ratio (DR) is the ratio of the EC₅₀ values of (S)-Glu in the presence and in the absence of a fixed antagonist concentration, [B].

(a) Adenylate Cyclase Activity

Adenylate cyclase activity is determined in initial experiments in transfected mammalian cells, using standard techniques. See, e.g., N. Adham, et al., supra,; R. L. Weinshank, et al. Proceedings of the National Academy of Sciences (USA), 89:3630-3634 (1992), and the references cited therein.

Mammalian cells (the cell line AV12-664 is especially preferred) are stably transfected with a plasmid comprising the cloned metabotropic glutmate receptor. The cells are maintained in a medium consisting of Dulbecco's Modified Eagle's Medium (DMEM) containing 5% dialyzed fetal calf serum, 10 mM HEPES buffer (pH 7.3), 1 mM sodium pyruvate, 1 mM glutamine, and 200 .μ.g/μΛ hygromycin.

For the assay the cells are disassociated from stock culture flasks with trypsin, and planted in 24-well plastic culture dishes (15 mm wells) at a density of 500,000-700,000 cells per well using the same culture medium. After twenty four hours incubation in a humidified carbon dioxide incubator, the cell monolayers are washed with buffer (Dulbecco's phosphate-buffered saline containing 0.5 mM isobutylmethylxanthine and 3 mM glucose) and then incubated in the same buffer at 37° C. for 30 minutes. The monolayers are then washed six additional times with buffer.

Drugs and forskolin, or forskolin alone, dissolved in buffer, are added after the final wash. After incubating for 20 minutes at 37° C., 0.5 mL of 8 mM EDTA is added to each well. The plates are then placed in a boiling water bath for about four minutes. The supernatant fluids are then recovered from the wells and lyophilized. Cyclic AMP (cAMP) determinations are carried out on the lyophilized samples using commercially available radioimmunoassay kits, following the manufacturer's instructions. The cAMP level in wells containing drug are the compared to the forskolin controls.

(b) Phosphatidylinositol Assay

Phosphatidylinositol hydrolysis in clonal cell lines of AV12 harboring a plasmid expressing the cloned metabotropic glutamate receptor is measured in response to glutamate agonists as a functional assay for metabotropic glutamate receptor activity according to D. Schoepp, Trends in Pharmaceutical Sciences, 11:508, 1990.

Twenty four-well tissue-culture vessels are seeded with approximately 250,000 cells per well in Dulbecco's Minimal Essential Media (D-MEM) (in the absence of glutamic acid) which contained 2 mM glutamine and 10% dialyzed fetal calf serum. After 24 hours growth at 37° C. the media is removed and replaced with fresh media containing four microcuries of [³H]myoinositol per well and the cultures are incubated a further 16 to 20 hours. The media is then removed and the cells in each well are washed with serum free medium containing 10 mM lithium chloride, 10 mM myoinositol, and 10 mM HEPES (2.×1 mL washes). After the final wash, 0.5 mL of washing solution is added containing the appropriate concentrations of drugs and vehicles.

If the particular assay is also testing antagonists, a ten minute incubation is performed prior to agonist induction. Cells are incubated for about one hour at 37° C. in 95%:5% O₂:CO₂ or as appropriate for time course. The reactions are terminated by removing media and adding 1 mL of cooled 1:1 acetone:methanol followed by induction on ice for a minimum of twenty minutes.

These extracts are then removed and placed in 1.5 mL centrifuge tubes. Each well is washed with 0.5 mL water and this wash is added to the appropriate extract. After mixing and centrifugation, each aqueous supernatant is processed by chromatography on a QMA SEP-PAK.™. column, which had previously been wetted and equilibrated by passing 10 mL of water, followed by 8 mL of IM triethylammonium hydrogen carbonate (TEAB), followed by 10 mL of water through the column.

The assay supernatants containing the water soluble [³H]inositol phosphate are passed over the columns. This is followed by a 10 mL water wash and a 4 mL wash with 0.02M TEAB to remove [H]inositol precursors. [³H]Inositol phosphate is eluted with 4 mL of 0.1M TEAB into scintillation vials and counted in the presence of scintillation cocktail. Total protein in each sample is measured using standard techniques. Assays are measured as the amount of [³H]inositol phosphate released per milligram of protein.

These types of assay, employing different subtypes of cloned metabotrapic receptors, may be used to determine which compounds have selective affinity in that they bind to one subtype of receptor with a greater affinity than another subtype. In performing such experiments with some of the compounds of the present invention, it has been demonstrated that some compounds of the present invention act as agonists with the cAMP-linked metabotropic glutamate receptors, while showing less activity with the phosphatidylinositol-linked metabotropic glutamate receptors.

Pharmaceutical Formulations

According to another aspect, the present invention provides a method of modulating one or more metabotropic glutamate receptor functions in a warm-blooded mammal which comprises administering an effective amount of a compound of Formula I, or a non-toxic metabolically-labile ester or amide thereof, or a pharmaceutically acceptable salt thereof.

The compounds of the present invention are preferably formulated prior to administration. Therefore, another aspect of the present invention is a pharmaceutical formulation comprising a compound of Formula I and a pharmaceutically-acceptable carrier, diluent, or excipient. The present pharmaceutical formulations are prepared by known procedures using well-known and readily available ingredients. In making the compositions of the present invention, the active ingredient will usually be mixed with a carrier, or diluted by a carrier, or enclosed within a carrier, and may be in the form of a capsule, sachet, paper, or other container. When the carrier serves as a diluent, it may be a solid, semi-solid, or liquid material that acts as a vehicle, excipient, or medium for the active ingredient.

The compounds of Formula I are usually administered in the form of pharmaceutical compositions. These compounds can be administered by a variety of routes including oral, rectal, transdermal, subcutaneous, intravenous, intramuscular, and intranasal. These compounds are effective as both injectable and oral compositions. Such compositions are prepared in a manner well known in the pharmaceutical art and comprise at least one active compound.

The present invention also provides pharmaceutical compositions containing compounds as disclosed in the claims in combination with one or more pharmaceutically acceptable, inert or physiologically active, diluent or adjuvant. The compounds of the invention can be freeze-dried and, if desired, combined with other pharmaceutically acceptable excipients to prepare formulations for administration. These compositions may be presented in any form appropriate for the administration route envisaged. The parenteral and the intravenous route are the preferential routes for administration.

Compounds of the general Formula I may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. In addition, there is provided a pharmaceutical formulation comprising a compound of general Formula I and a pharmaceutically acceptable carrier. One or more compounds of general Formula I may be present in association with one or more non-toxic pharmaceutically acceptable carriers and/or diluents and/or adjuvants and if desired other active ingredients. The pharmaceutical compositions containing compounds of general Formula I may be in a form suitable for oral use, for example, as tablets, troches, lozenges, aqueous or oily suspensions, dispersible powders or granules, emulsions, hard or soft capsules, or syrups or elixirs.

Compositions intended for oral use may be prepared according to any known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate: granulating and disintegrating agents for example, corn starch, or alginic acid: binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain active materials in admixture with excipients suitable for the manufacture of aqueous suspensions. Such excipients are suspending agents, for example sodium carboxylmethylcellulose, methyl cellulose, hydropropylmethylcellulose, sodium alginate, polyvinylpyrrolidone, gum tragacanth and gum acacia: dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example hepta-decaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl-p-hydroxy benzoate, one or more coloring agents, one or more flavoring agents or one or more sweetening agents, such as sucrose or saccharin.

Oily suspensions may be formulated by suspending the active ingredients in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the invention may also be in the form of oil-in-water emulsions. The oil phase may be a vegetable oil, for example olive oil or peanut oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to known art using those suitable dispersing or wetting agents and suspending agents that have been mentioned above. The sterile injectable preparation may also be a sterile injectable solution or a suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compound(s) of the general Formula I may be administered, together or separately, in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compound(s) of general Formula I may be administered, together or separately, parenterally in sterile medium. The drug, depending on the vehicle and concentration used, can either be suspended or dissolved in the vehicle. Advantageously, adjuvants such as local anaesthetics, preservatives and buffering agents can be dissolved in the vehicle.

The dosage to be administered is not subject to defined limits, but it will usually be an effective amount. It will usually be the equivalent, on a molar basis of the pharmacologically active free form produced from a dosage formulation upon the metabolic release of the active free drug to achieve its desired pharmacological and physiological effects. The compositions are preferably formulated in a unit dosage form, each dosage containing from about 0.05 to about 100 mg, more usually about 1.0 to about 30 mg, of the active ingredient. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient.

The active compound is effective over a wide dosage range. For examples, dosages per day normally fall within the range of about 0.01 to about 30 mg/kg of body weight. A typical daily dose will contain from about 0.01 mg/kg to about 100 mg/kg of the active compound of this invention. Preferably, daily doses will be about 0.05 mg/kg to about 50 mg/kg, more preferably from about 0.1 mg/kg to about 25 mg/kg. In the treatment of adult humans, the range of about 0.1 to about 15 mg/kg/day, in single or divided dose, is especially preferred. However, it will be understood that the amount of the compound actually administered will be determined by a physician, in the light of the relevant circumstances, including the condition to be treated, the chosen route of administration, the actual compound administered, the age, weight, and response of the individual patient, and the severity of the patient's symptoms, and therefore the above dosage ranges are not intended to limit the scope of the invention in any way. In some instances dosage levels below the lower limit of the aforesaid range may be more than adequate, while in other cases still larger doses may be employed without causing any harmful side effect, provided that such larger doses are first divided into several smaller doses for administration throughout the day.

The compositions are preferably formulated in a unit dosage form, each dosage containing from about 5 mg to about 500 mg, more preferably about 25 mg to about 300 mg of the active ingredient. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical carrier, diluent, or excipient. The following formulation examples are illustrative only and are not intended to limit the scope of the invention in any way.

Formulation 1

Hard gelatin capsules are prepared using the following ingredients:

| | Quantity (mg/capsule) |
|---|---|
| Active Ingredient | 250 |
| Starch, dried | 200 |
| Magnesium stearate | 10 |
| Total | 460 |

The above ingredients are mixed and filled into hard gelatin capsules in 460 mg quantities.

Formulation 2

A tablet is prepared using the ingredients below:

| | Quantity (mg/tablet) |
|---|---|
| Active Ingredient | 250 |
| Cellulose, microcrystalline | 400 |
| Silicon dioxide, fumed | 10 |
| Stearic acid | 5 |
| Total | 665 |

The components are blended and compressed to form tablets each weighing 665 mg.

Formulation 3

An aerosol solution is prepared containing the following components:

| | Weight % |
|---|---|
| Active Ingredient | 0.25 |
| Ethanol | 29.75 |
| Propellant 22 (Chlorodifluoromethane) | 70.00 |
| Total | 100 |

The active compound is mixed with ethanol and the mixture added to a portion of the Propellant 22, cooled to −30° C. and transferred to a filling device. The required amount is then fed to a stainless steel container and diluted with the remainder of the propellant. The valve units are then fitted to the container.

Formulation 4

Tablets each containing 60 mg of active ingredient are made as follows:

| | Quantity (mg/tablet) |
|---|---|
| Active Ingredient | 60 |
| Starch | 45 |
| Microcrystalline cellulose | 35 |
| Polyvinylpyrrolidone | 4 |
| Sodium carboxymethyl starch | 4.5 |
| Magnesium stearate | 0.5 |
| Talc | 1.0 |
| Total | 150 |

The active ingredient, starch, and cellulose are passed through a No. 45 mesh U.S. sieve and mixed thoroughly. The solution of polyvinylpyrrolidone is mixed with the resultant powders that are then passed through a No. 14 mesh U.S. sieve. The granules so produced are dried at 50° C. and passed through a No. 18 mesh U.S. sieve. The sodium carboxymethyl starch, magnesium stearate, and talc, previously passed through a No. 60 mesh U.S. sieve, are then added to the granules which, after mixing, are compressed on a tablet machine to yield tablets each weighing 150 mg.

Formulation 5

Capsules each containing 80 mg medicament are made as follows:

| | Quantity (mg/capsule) |
|---|---|
| Active Ingredient | 80 |
| Starch | 59 |
| Microcrystalline cellulose | 59 |
| Magnesium stearate | 2 |
| Total | 200 |

The active ingredient, cellulose, starch, and magnesium stearate are blended, passed through a No. 45 sieve, and filled into hard gelatin capsules in 200 mg quantities.

Formulation 6

Suppositories each containing 225 mg of active ingredient may be made as follows:

| | Quantity (mg/suppository) |
|---|---|
| Active Ingredient | 225 |
| Saturated fatty acid glycerides | 2000 |

Total 2225

The active ingredient is passed through a No. 60 mesh U.S. sieve and suspended in the saturated fatty acid glycerides previously melted using the minimum heat necessary. The mixture is then poured into a suppository mold of nominal 2 g capacity and allowed to cool.

Formulation 7

Suspensions each containing 50 mg of medicament per 5 mL dose are made as follows:

| | | |
|---|---|---|
| Active Ingredient | 50 | mg |
| Sodium carboxylmethyl cellulose | 50 | mg |
| Syrup | 1.25 | mL |
| Benzoic acid solution | 0.10 | mL |
| Flavour | q.v. | |
| Color | q.v. | |
| Purified water to total | 5 | mL |

The medicament is passed through a No. 45 mesh U.S. sieve and mixed with the sodium carboxymethyl cellulose and syrup to form a smooth paste. The benzoic acid solution, flavor and color are diluted with some of the water and added, with stirring. Sufficient water is then added to produce the required volume.

Formulation 8

An intravenous formulation may be prepared as follows:

Quantity

| | | |
|---|---|---|
| Active Ingredient | 100 | mg |
| Mannitol | 100 | mg |
| 5 N Sodium hydroxide | 200 | mL |
| Purified water to total | 5 | mL |

Formulation 9

A topical formulation may be prepared as follows:

Quantity

| | | |
|---|---|---|
| Active Ingredient | 1-10 | g |
| Emulsifying Wax | 30 | g |
| Liquid Paraffin | 20 | g |
| White soft paraffin to | 100 | g |

The white soft paraffin is heated until molten. The liquid paraffin and emulsifying wax are incorporated and stirred until dissolved. The active ingredient is added and stirring is continued until dispersed. The mixture is then cooled until solid.

Formulation 10

Sublingual or buccal tablets, each containing 10 mg of active ingredient, may be prepared as follows:

| | Quantity (mg/tablet) |
|---|---|
| Active Ingredient | 10.0 |
| Glycerol | 210.5 |
| Water | 143.0 |
| Sodium Citrate | 4.5 |
| Polyvinyl Alcohol | 26.5 |
| Polyvinylpyrrolidone | 15.5 |
| Total | 410.0 |

The glycerol, water, sodium citrate, polyvinyl alcohol, and polyvinylpyrrolidone are admixed together by continuous stirring and maintaining the temperature at about 90° C. When the polymers have gone into solution, the solution is cooled to about 50°-55° C. and the medicament is slowly admixed. The homogenous mixture is poured into forms made of an inert material to produce a drug-containing diffusion matrix having a thickness of about 2-4 mm. This diffusion matrix is then cut to form individual tablets having the appropriate size.

Another preferred formulation employed in the methods of the present invention employs transdermal delivery devices (“patches”). Such transdermal patches may be used to provide continuous or discontinuous infusion of the compounds of the present invention in controlled amounts.

The construction and use of transdermal patches for the delivery of pharmaceutical agents is well known in the art (see, for example, U.S. Pat. No. 5,023,252, issued Jun. 11, 1991) herein incorporated by reference. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. One such implantable delivery system, used for the transport of biological factors to specific anatomical regions of the body, is described in U.S. Pat. No. 5,011,472, issued Apr. 30, 1991, which is herein incorporated by reference.

Indirect techniques, which are generally preferred, usually involve formulating the compositions to provide for drug latentiation by the conversion of hydrophilic drugs into lipid-soluble drugs or prodrugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic. drugs may be enhanced by intra-arterial infusion of hypertonic solutions that can transiently open the blood-brain barrier.

### EXAMPLES

The following Examples illustrate the invention. The following abbreviations are used in the Examples: EtOAc, ethyl acetate; THF, tetrahydrofuran; EtOH, ethanol; TLC, thin layer chromatography; GC, gas chromatography; HPLC, high pressure liquid chromatography; m-CPBA, m-chloroperbenzoic acid; Et₂O, diethyl ether; DMSO, dimethyl sulfoxide; DBU, 1,8-diazabicyclo-[5.4.0]undec-7-ene, MTBE, methyl t-butyl ether; and FDMS, field desorption mass spectrometry.

### Example 1

### 1-Methyl-5-glycyl barbituric Acid (4)

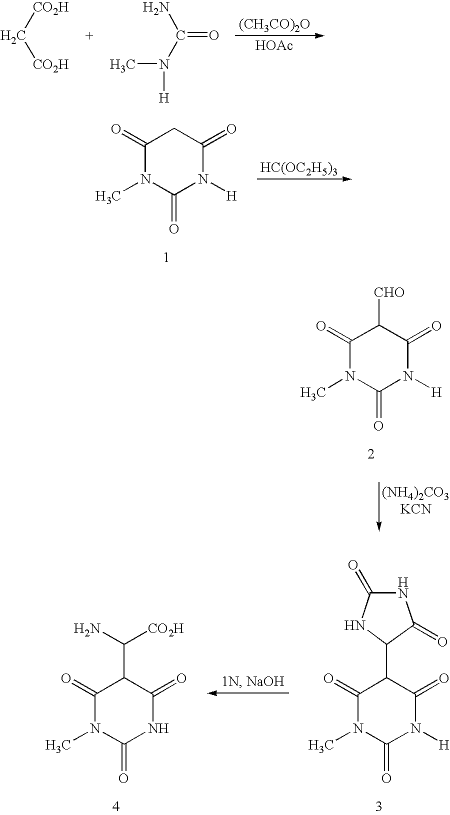

5 g (0.068 mol) of 1-methylurea and 8 g (0.079 mol) of dry malonic acid were dissolved in 18 mL of glacial acetic acid at 60-70° C. The resulting solution was treated with 13 mL (0.13 mol) of acetic anhydride in 1.3 h, and raised the temperature rapidly to 90° C. After keeping the temperature at 90° C. for 3 h, the solution was concentrated in vacuum. To the resulting syrup, 10 mL of ethanol was added and the mixture was warmed slowly until the solution occurs. On cooling, 1-methylbarbituric acid (1) was formed in 50% yield. (mp 130-132).

0.05 mol of 1-methylbarbituric acid (1) was refluxed in 60 mL of ethyl orthoformate, with stirring for 1.5 h. On cooling, crystals were formed that were collected by filtration. Recrystalization from ethanol yielded 75% of 1-methyl-5-formylbarbituric acid (2), (mp 197-199° C.) (decomposed).

4 g (0.04 mol) of ammonium carbonate and 1.3 g (0.02 mol) of potassium cyanide were added to a suspended solution of 1.8 g (0.01 mol) of 1-methyl-5-formylbarbituric acid in 25 mL of water. The resulting solution was stirred at 55-65° C. for 5 h, and refluxed overnight. The resulting yellowish mixture was allowed to cool to room temperature and acidified with 6N HCl to pH 4. Upon evaporation in vacuo to dryness, the precipitates were formed.

The solid formed above was dissolved in 100 mL of IN NaOH to give a deep red colored solution that was refluxed for 6 hours. After cooling, the resulting solution was acidified to pH 2.5 with 6N HCl. After evaporation, the residue was redissolved in 5 ML of H₂O and placed on a Spectrum×4 OH⁻ anion exchange resin until yield was obtained. Column elution with 0.5 N, acetic acid, and evaporation of the solvent, resulted in the title compound in 15% yield. Analysis calculated for C₇H₉N₃O₅+NH₄Cl.H₂O: % C, 28.96; %H, 4.82; %N, 17.86. Found: %C, 29.48; %H, 5.11; %H, 16.42.

¹H NMR (200 MHz, D₂O) δ 1.50 (s, 3H), 2.2 (t, 2H), 3.2 (t, 1H), COOH and NH₂ protons not observed.

### Example 2

### 5[2-Amino-2-carboxypropyl]barbituric Acid (7)

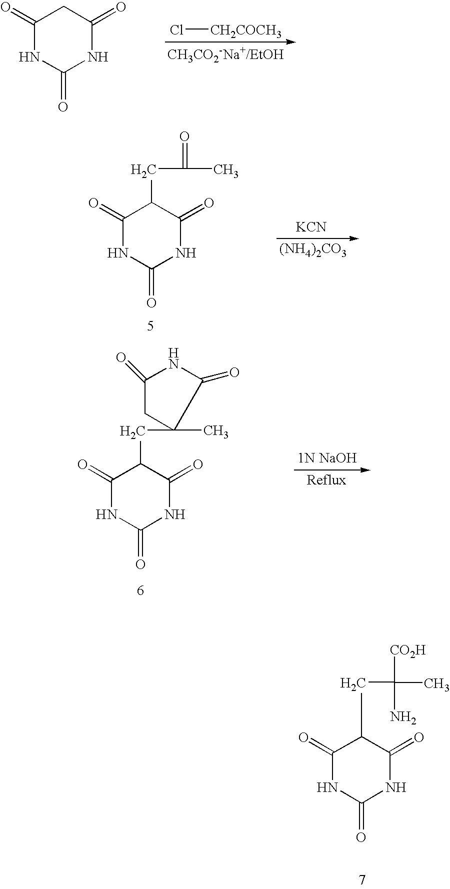

12.5 g of barbituric acid was dissolved in 75 cc of hot water. To this solution, 10.37 g of crystalline sodium acetate and 7.05 g of chloroacetone in 75 cc of 80% ethanol was added gradually. The aqueous solution of barbituric acid was kept boiling under a reflux condenser while the alcoholic solution was added from a dropping funnel during the course of one hour. A bright yellow colour gradually developed and at the same time a yellow precipitates were formed. After all of the reagent had been added the refluxing was continued for two hours. As the mixture was cooled, yellow precipitates separated and were isolated in 80% yield. Purification was performed by suspending the solid in 75 cc of cold water, adding 10% sodium hydroxide to exact neutrality and filtering from a small residue. The filtrate was acidified with HCl to give acetonyl barbituric acid (5) as a yellow solid.

1.84 g of the yellow solid formed above, was suspended in 20 cc of water and treated with 3.84 g of ammonium carbonate. To the resulting clear orange solution 1.30 g of potassium cyanide was added and stirred at 60-65° C. for eight hours and refluxed overnight. The solution was evaporated to dryness and the resulting solid was used straight for the next step.

To the above formed orange solid (6) 100 cc of 1 N NaOH was added. The solution was refluxed overnight, then acidified with 6 N HCl to pH 2. After evaporation, the salt was taken up into absolute ethanol and the unwanted salt was filtered off. To the alcoholic filtrate, propylene oxide was added and the resulting solution was evaporated to give the title compound (7) in 22% yield.

Analysis calculated for C₈H₁₁N₃O₅: % C, 31.75; %H, 4.98; %N, 17.01. Found: %C, 31.25; %H, 5.45; %H, 17.02.

¹H NMR (200 MHz, D₂O) δ 1.9 (d, 1H), 2.95 (d, 3H), 3.5 (s, 3H), COOH and NH₂ protons not observed.

### Example 3

### 2-Amino, 2-(1-methyl, 2, 4, 6, trioxo-5-carboxypropylperihydropyrimindin-5-yl) Pimelic Acid (II)

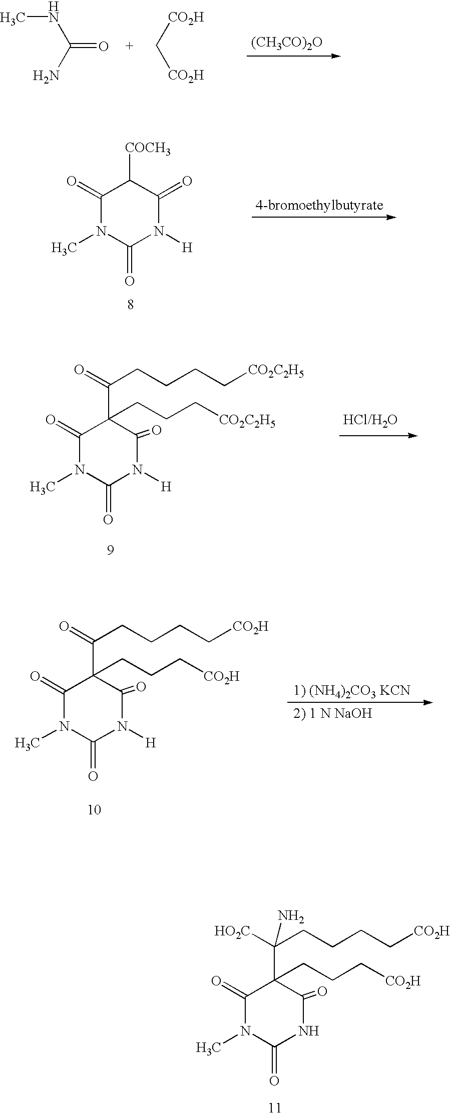

5 g (0.068 mol) of methyl urea and 8 g (0.079 mol) of dry malonic acid were dissolved in 20 mL of acetic anhydride. To this solution, 10 drops of concentrated H₂SO₄ were added and heated at 80° C. for 1 hour. Then temperature was raised to the boiling point and heated for additional three hours. Cooling of the resulting solution to room temperature followed by addition of ethanol resulted in the formation of yellow precipitates of compound (8) that were filtered (mp 209° C.).

To a suspended solution of 1 gram of NaH (60% dispersion in mineral oil) in 12 mL of dry THF, a solution of 3.35 g of the compound (8) in 50 mL DMF was added. After stirring for 3 hours, 4 g (3 mL) of 4-bromo-ethylbutyrate was added, followed by addition of 0.5 g of KI. The mixture was refluxed overnight then cooled to room temperature and extracted with ethylacetate and dried over MgSO₄ to give compound (9) as a solid, which was used for the next step without purification.

The compound (9) formed above, was suspended in a solution of 30 mL of 10% HCl and 30 mL of dioxane. The resulting mixture was refluxed for 6 hours to give a dark solution. The solution was neutralized with 10% NaOH to pH 7 and extracted with EtOAC and dried over MgSO₄ and concentrated in vacuo to give a compound (10) as a solid, that was used in the next reaction without purification.

1.4 g of compound (10) was dissolved in 50 cc of EtOH-H₂O and treated with 1 g of KCN and 3 g of ammonium carbonate. The mixture was heated at 60-70° C. overnight. After acidification to pH 2, the solution was evaporated to dryness. To this solid 100 cc of 1 N NaOH was added and refluxed overnight. After acidification to pH 2, the solution is evaporated to dryness. Absolute ethanol was added and the salt was filtered off. Propylene oxide (20 mL) was added to the filtrate and evaporated until a sticky solid remained. The residue was run through a cation exchange resin and elution was performed with 0.5 N, NH₄OH, and the title compound (11) was collected as a white solid in 30% yield.

¹H NMR (200 MHz, DMSO) δ 2.60 (s, 3H), 2.40-2.20 (m, 6H), 1.6-1.0 (m, 8H).

8. (Amended) A [pharmaceutical] composition, comprising a pharmaceutically acceptable carrier, diluent or excipient and a compound formula (I): 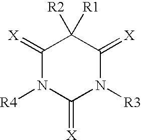
or stereoisomers thereof or pharmaceutically acceptable salts or hydrates thereof, wherein:
R1 and R2 can be the same or different and selected from the group comprising H, NH₂, COOH and (CH₂)nCR5WY, wherein: n=0-5, W is H or COOH, Y is H or NH₂, R5 is H, alkyl, aryl, or (CH₂)mR6, wherein: m=0-5 and R6 is H, carboxyl, phosphono, phosphino, sulfono, sulfino, borono, tetrazol, or isoxazol;
R3 and R4 can be the same or different and selected from the group comprising H, alkyl, aryl, acyl, CH₂COOH, NH₂ and —(CH₂)n′CHCO₂H NH₂, wherein: n′ is 0-5;
and X is O or S;
with the proviso, that at least one of R1, R2, R3 or R4 comprises an NH₂ moiety and at least one of R1, R2, R3 or R4 comprises one COOH moiety.

9. (Amended) A [use of the pharmaceutical composition according to claim 8,] method for modulating one or more metabotropic glutamate receptor functions in a warm blooded [mammals,] mammal, wherein said [use] method comprises administrating [of] to said mammal an effective amount of said [pharmaceutical] composition according to claim 8.

10. (Amended) A method [use of the pharmaceutical composition according to claim 8,] for treating a neurological disease or disorder selected from the group comprising: cerebral deficits subsequent to cardiac bypass surgery and grafting, cerebral ischemia, stroke, cardiac arrest, spinal cord trauma, head trauma, perinatal hypoxia, and hypoglycemic neuronal damage, Alzheimer's disease, Huntington's Chorea, amyotrophic lateral sclerosis, AIDS-induced dementia, ocular damage, retinopathy, cognitive disorders, idiopathic and drug-induced Parkinson's disease, muscular spasms, convulsions, migraine headaches, urinary incontinence, psychosis, drug tolerance, withdrawal, and cessation (i.e. opiates, benzodiazepines, nicotine, cocaine, or ethanol), smoking cessation, anxiety and related disorders (e.g. panic attack), emesis, brain edema, chronic pain, sleep disorders, Tourette's syndrome, attention deficit disorder, and tardive dyskinesia, wherein said [use] method comprises administrating [of] an effective amount of said [pharmaceutical] composition according to claim 8.

11. (Amended) A method [use of the pharmaceutical composition according to claim 8, in] for treating a psychiatric disease or disorder selected from the group comprising: schizophrenia, anxiety and related disorders (e.g. panic attack), depression, bipolar disorders, psychosis, and obsessive compulsive disorders, wherein said [use] method comprises administrating [of] an effective amount of said [pharmaceutical] composition according to claim 8.

12. (Amended) The [use] method according to any one of claims 9, 10 or 11, wherein said compound is selected from the group of compounds comprising

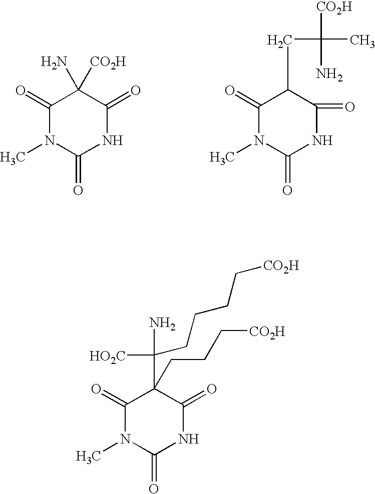

13. (Amended) A method of modulating the activity of metabotropic glutamate receptors in a mammal, comprising administering to a mammal requiring modulated excitatory amino acid neurotransmission, a therapeutically effective amount of the [pharmaceutical] composition according to claim 8.
